(19) United States
(12) Patent Application Publication
(10) Pub. No.: US 2012/0123366 A1
(43) Pub. Date: May 17, 2012

(54) REPLACEABLE ABSORBENT INSERT FOR USE IN TWO-PIECE DIAPERING SYSTEM WITH RE-USABLE DIAPER SHELL

(76) Inventor: James Roy Brownlee, West Vancouver (CA)

(21) Appl. No.: 13/143,977

(22) PCT Filed: Jan. 12, 2010
(86) PCT No.: PCT/CA10/00050

§ 371 Date: Sep. 22, 2011

Related U.S. Application Data
(60) Provisional application No. 61/144,145, filed on Jan. 12, 2009.

(51) Int. Cl. A61F 13/505; A61L 15/26

(52) U.S. Cl. 604/369; 604/385.14

(57) ABSTRACT

A two-piece disposable diapering system is disclosed which has a re-usable, washable diaper shell and a replaceable absorbent insert having an absorbent pad narrower than the space between the opposed leg cuffs of the shell whereby when fitted to the wearer the central channel region of the diaper is significantly deeper than the height of the opposed leg cuffs.

### CROSS REFERENCE TO RELATED APPLICATION

The present application claims the benefits, under 35 U.S.C.§119(e), of U.S. Provisional Application Ser. No. 61/144,145 filed Jan. 12, 2009 entitled “Replaceable Absorbent Insert For Use In Two-Piece Diapering System With Re-usable Diaper Shell” and is a continuation-in-part of U.S. patent application Ser. No. 12/375,172 filed Aug. 2, 2007 entitled “Diapering System Using Re-usable Diaper Shell With Replaceable Absorbent Insert And Method of Manufacture of Same” which are incorporated herein by this reference.

### TECHNICAL FIELD

The invention relates to the construction of infant diapers, and particularly to disposable diapers having a re-usable, washable shell and a replaceable absorbent insert.

### BACKGROUND

Currently most infant diapers are of the disposable variety. Machine washable cloth diapers are unpopular due to the expense, time and labor required to wash them and are more expensive in initial cost. Existing disposable diapers have a liquid impervious outer layer, an inner non-woven liner and an integral layer of absorbent material, typically pulp fluff, sandwiched between the inner and outer layers. Disposable diapers are not re-usable, washable or recyclable. They create a large volume of waste, since the entire garment is disposed of after a single use. If the diaper is wetted or soiled even slightly, the entire diaper is discarded, at considerable expense and causing considerable waste.

Currently some disposable diapers design use a rectangular shell which when fitted to the infant forms a deeper central channel which contains waste more effectively than previous designs.

There is a need for an infant diaper design which reduces the amount of waste, is re-usable, rinsable and/or washable and recyclable, and has the design advantages of current deep channel disposable diapers.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Consequently, the present invention provides a two-piece diaper system having a re-usable shell and a removable and replaceable absorbent pad in which the absorbent pad is significantly narrower than the space between the leg gathers to provide a deep central channel when worn. The re-usable diapering system of the invention consists of a non-absorbent outer liquid impervious shell, as in a standard disposable diaper, and may have adhesive closures and elasticized legs, but without an integral central absorbent layer which is exposed to liquids in the crotch area. Rather, a separate disposable absorbent pad is provided in the central crotch area of the disposable diaper which is not sandwiched in the shell. The central area of the shell is stiffened to facilitate manufacture and handling by the user. The absorbent pad is significantly narrower than the space between the opposed leg cuffs whereby when fitted to the wearer said central channel region is significantly deeper than the height of said opposed leg cuffs. The absorbent pad may have protective waterproof covers which extend over the leg gathers and may be removably secured by a releasable adhesive, in order to facilitate cleaning and reusing the shell.

The invention provides a removable, replaceable absorbent insert for use with a re-usable diaper shell, the shell comprising: i) a pliant non-absorbent, liquid impervious body for removable fitting to the wearer, forming an interior and an exterior surface, a front and back portion, opposed side edges and a crotch area when so fitted; ii) opposed, non-absorbent liner portions secured along the opposed side edges of the body and forming opposed elastically contractible leg cuffs extending between the front and back portion, the opposed elastically contractible leg cuffs thereby defining a central region between the opposed leg cuffs extending between the front and back portion, the central channel region being without absorbent material which is exposed to liquid in the crotch area and comprising in the central channel region flexible means for stabilizing said diaper shell. The removable, replaceable absorbent insert comprises an absorbent pad comprising a body of absorbent material having a non-woven hydrophilic covering, the insert being sized and shaped to be retained adjacent the interior surface of the diaper shell in the central region, and wherein the absorbent pad is significantly narrower than the space between the opposed leg cuffs.

According to one aspect of the invention, the replaceable insert has attached hydrophobic covers extending from opposed sides of the pad, The protective covers may have elongated elastic members secured therein to form leg gathers when in operative position and to assist in lifting the protective covers over the leg gathers of the shell.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. It is intended that the embodiments and figures disclosed herein are to be considered illustrative rather than restrictive.

In drawings which disclose a preferred embodiment of the invention:

FIG. 1 is a perspective view of a prior art conventional hourglass-shaped disposable infant diaper;

FIG. 2 is a cross-section taken along line A-A of FIG. 2A (not to scale);

FIG. 2A is a top view of a current prior art rectangular deep channel diaper design;

FIG. 3 is a perspective view of a re-usable non-absorbent infant diaper shell for use with the invention;

FIG. 4A is a cross-section taken along line 4-4 of FIG. 3 (not to scale);

FIG. 4B is a cross-section taken along line 4-4 of FIG. 3 (not to scale) showing a first method of attaching a stabilizing layer;

FIG. 4C is a cross-section taken along line 4-4 of FIG. 3 (not to scale) showing a second method of attaching a stabilizing layer;

FIG. 4D is a cross-section taken along line 4-4 of FIG. 3 (not to scale) showing a third method of attaching a stabilizing layer;

FIG. 4E is a cross-section taken along line 4-4 of FIG. 3 (not to scale) showing a fourth method of attaching a stabilizing layer;

FIG. 5 is a perspective view of the removable/replaceable absorbent pad of the invention;

FIG. 6 is a cross-section illustrating the replaceable absorbent pad in a first configuration in the re-usable shell of FIG. 3 (not to scale) taken along lines 4-4;

FIG. 7 is a cross-section illustrating the replaceable absorbent pad in a second configuration in the re-usable shell of FIG. 3 (not to scale)) taken along lines 4-4;

FIG. 8 is a cross-section (not to scale) illustrating the replaceable absorbent pad in the re-useable shell of FIG. 9, taken along lines 8-8;

FIG. 9 is a is a perspective view of a second embodiment of the re-usable non-absorbent infant diaper shell of the invention;

FIG. 10 is a perspective view (not to scale) illustrating a further embodiment of the replaceable absorbent pad without wings;

FIG. 11 is a perspective view (not to scale) illustrating a further embodiment of the replaceable absorbent pad with wings and elastic members with deeper gathers;

FIG. 12 is a perspective view (not to scale) illustrating a further embodiment of the replaceable absorbent pad without elastic members;

FIG. 13 is a perspective view (not to scale) illustrating a further embodiment of the replaceable absorbent pad with wings and shallow channel;

FIG. 14 is a perspective view (not to scale) illustrating a further embodiment of the replaceable absorbent pad with wings and deep channel;

FIG. 15 is a perspective view (not to scale) illustrating a further embodiment of the replaceable absorbent pad with a pad that curves to snuggly fit the shell;

FIG. 16 is a perspective view (not to scale) illustrating a further embodiment of the replaceable absorbent pad with a pad that has bends to snugly fit the shell;

FIG. 17 is a perspective view (not to scale) illustrating a further embodiment of the replaceable absorbent pad without wings or leg gathers;

FIG. 18 is a perspective view (not to scale) illustrating a further embodiment of the replaceable absorbent pad with wings and elastic members with deeper gathers provided with additional stacked pads; and

FIG. 19 is a perspective view (not to scale) illustrating a further embodiment of the replaceable absorbent pad with elastic members on the underside of the pad.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein the term “washable” means that the article can be re-used after washing by hand.

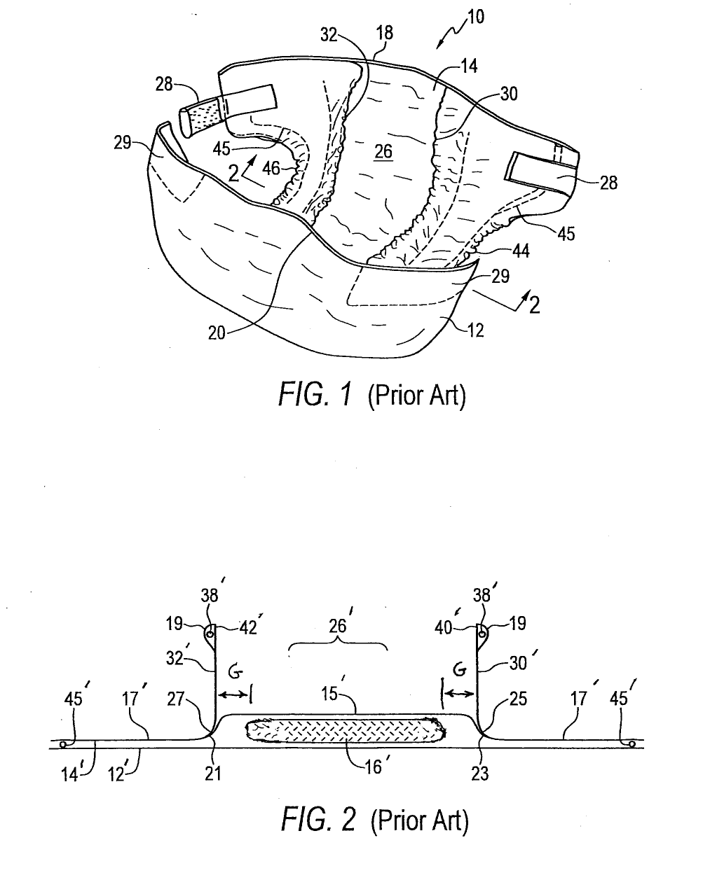

FIGS. 1, 2 and 2A illustrate conventional disposable diapers 10, 10′. Referring to FIG. 1, diaper 10 is constructed of an hour-glass shaped backsheet 12 made of a lightweight liquid-impervious polyethylene plastic, a non-woven liner 14 made from a non-absorbent synthetic plastic such as non-woven polypropylene, and an absorbent pad 16 sandwiched between backsheet 12 and liner 14. The diaper has a back waist section 18, front waist section 20, and crotch area 26. Adhesive flaps 28 are provided to attach the garment around the infant. Areas 29 of high gloss polyethylene plastic film are provided on backsheet 12 to which adhesive strips 28 can releasably adhere without tearing the backsheet 12 on removal. Alternatively flaps 28 and sections 29 may comprise hook and loop fasteners. Elasticized interior leg gathers 30, 32 are formed in the non-woven liner with elastic members running along inner edges as shown as 38′ in FIG. 2. Outside elastic leg cuffs are provided at 44, 46 with elastic members 45 provided at their outer edges. Elastic members 38′, 45 are typically thin rubber ribbons, or a hot melt elastomeric adhesive may be used as is known in the disposable diaper art.

As illustrated in FIGS. 2 and 2A, a prior art rectangular disposable one-piece diaper is constructed of a rectangular backsheet 12′ made of a lightweight liquid-impervious polyethylene plastic, a non-woven liner 14′ made from a non-absorbent synthetic plastic such as non-woven polypropylene, and an absorbent pad 16′ sandwiched between backsheet 12′ and liner 14′. The diaper has a crotch area 26′. Hook fasteners are provided on flaps 28′ to attach the garment around the infant by releasably attaching to areas of loop-functioning plastic film provided on backsheet 12′ to which flaps 28′ can releasably adhere without tearing the backsheet 12′ on removal. Elasticized interior leg gathers 30′, 32′ are formed in the non-woven liner with elastic members 38′ running along inner edges 40′, 42′. Outside elastic leg cuffs are provided with elastic members 45′ provided at the outer edges of body 12′. Elastic members 38′, 45′ are typically thin rubber ribbons, or a hot melt elastomeric adhesive may be used as is known in the disposable diaper art.

The non-woven liner 14′ of the conventional rectangular disposable diaper comprises three sections, an inner hydrophilic sheet 15′ and outer hydrophobic sheets 17′. Sheet 15′ permits moisture to penetrate to pad 16′ while keeping the skin of the infant away from the pad 16′. Sheets 17′ form the leg gathers 30′, 32′ which retain fecal matter and moisture in the central crotch area channel 26′. Absorbent pad 16′ is formed of pulp fluff material which is wrapped in a thin layer of absorbent paper tissue to maintain the integrity of the pad and prevent bunching of the fluff air-laid technology can also be used to reduce the weight of pulp, volume, thickness of the pad and to avoid SAP particles from contacting the baby's skin. Crystals of super absorbent polymers are typically distributed throughout the fluff to increase the absorbency of the pad 16′.

Since a large gap “G” is left between the edges of pad 16′ and the base of leg gathers 30′, 32′, when the one-piece conventional diaper 10′ is fitted on the infant, the contraction of elastic elements 40′, 42′ causes the depth of the channel 26′ to be increased by the width of gaps G. This creates a deeper channel for retaining fecal matter, causing this diaper design to be more effective at avoiding leakage.

In the existing manufacturing process, the two outer hydrophobic sheets 17′ are first typically formed by slicing a tensioned sheet of the material and then forming outward folds 19 around elastic members 38′. Sheet 15′ is then glued or heat welded at either edge 21, 23 to outer sheets 17′ along lines 25, 27. In a continuous process, non-woven liner 14′, backsheet 12′ and pad 16′ are then glued or heat welded into a single unitary sandwich, with glue typically applied to the upper surface of backsheet 12′ to secure pad 16′ and liner 17′. A few small spaced drops of glue are also applied to the top of pad 16′ in spaced locations to attach it to sheet 15′ and prevent movement of the pad 16′.

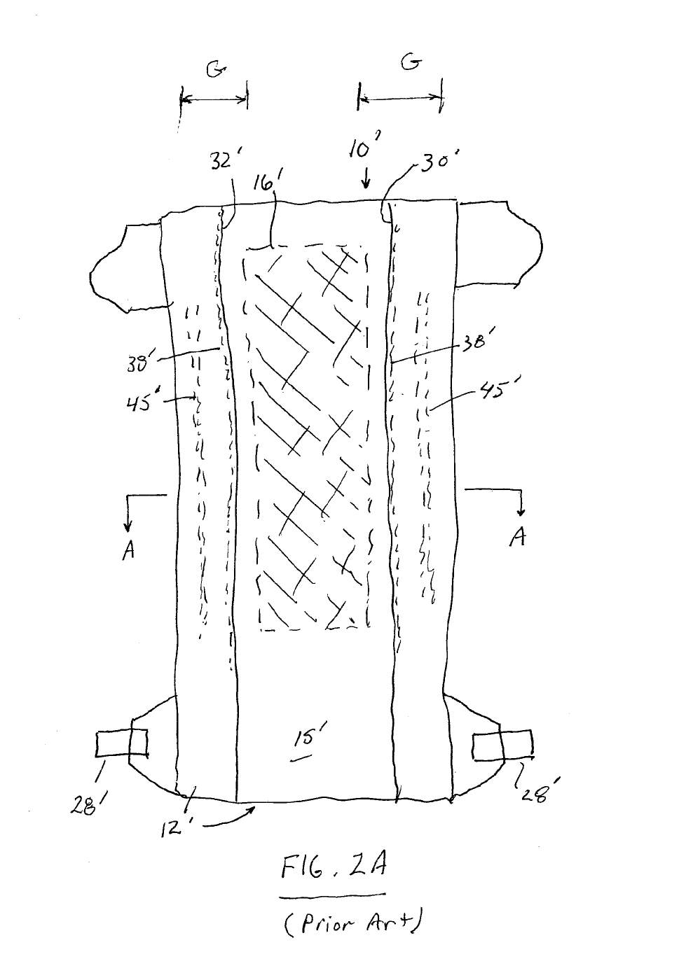

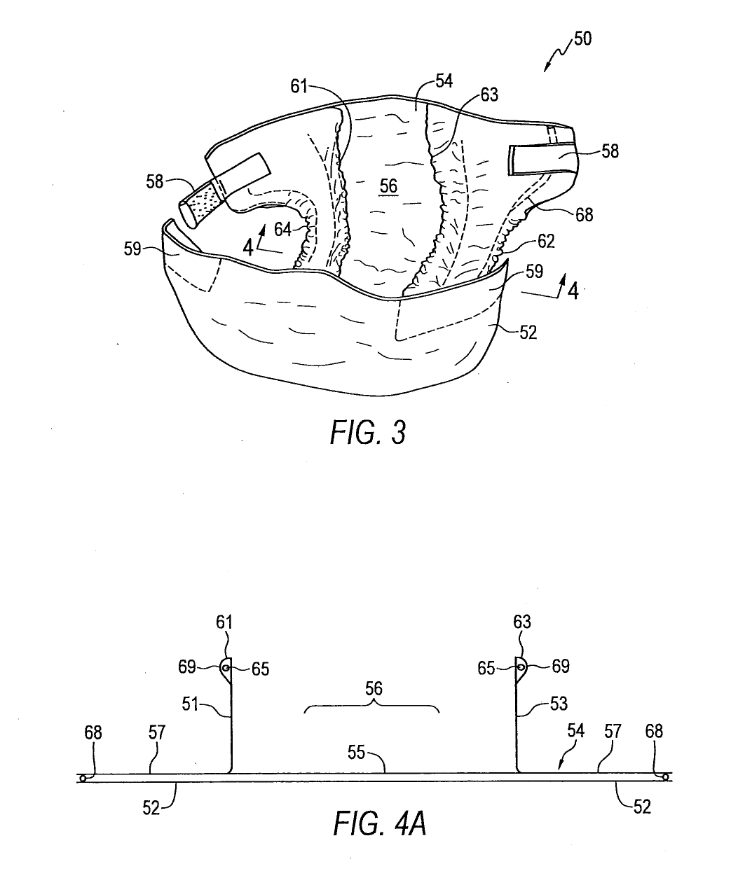

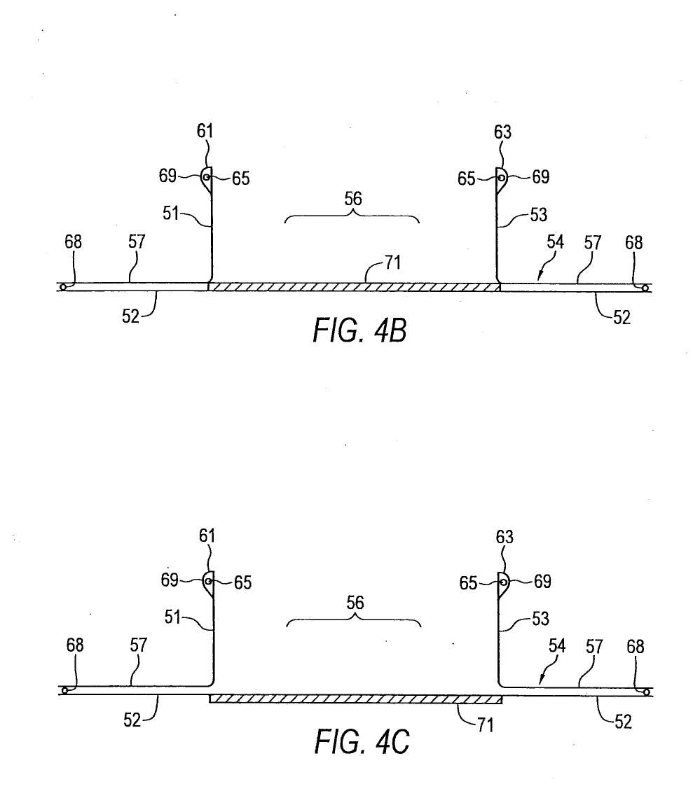

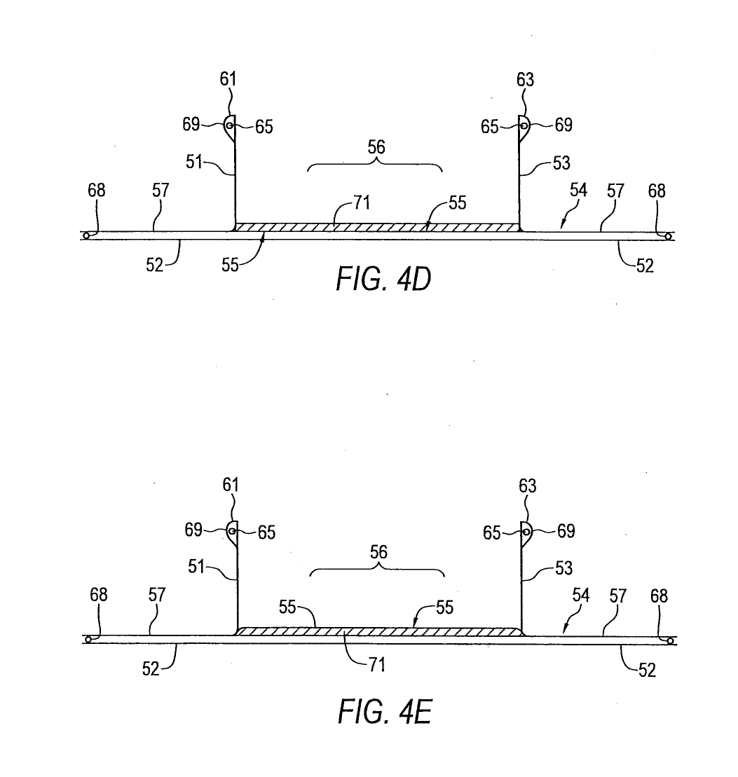

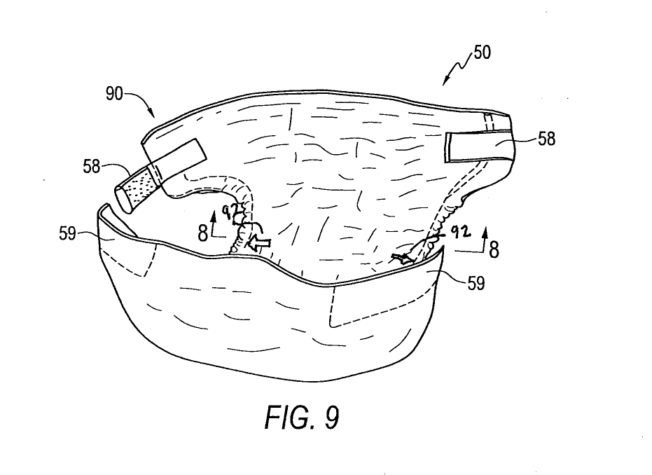

A washable, re-usable diaper shell 50 for use with the present invention is shown in FIGS. 3 and 4. It can be either hourglass shaped as in FIG. 1 or rectangular as in FIG. 2A. The water-impermeable back sheet 52 and water impermeable liner sheet 54 are formed in a manner similar to the conventional disposable diaper 10, using conventional disposable diaper machinery for manufacturing disposable diapers, however no absorbent pad or material 16 is exposed to the interior in the crotch area 56. Instead, the inner layer 55 in crotch area 56 of liner sheet 54 may be a layer of waterproof plastic or non-woven, which is preferably stiffer and less pliant than the backer 52. Elasticized leg cuffs 62, 64 are formed by providing elastic members 68 between backer 52 and liner sheets 57. Interior leg gathers 61, 63 are formed with elastic members 65 within folds 69. The embodiment shown in FIG. 9 uses the same construction except no internal leg gathers 61, 63 are provided.

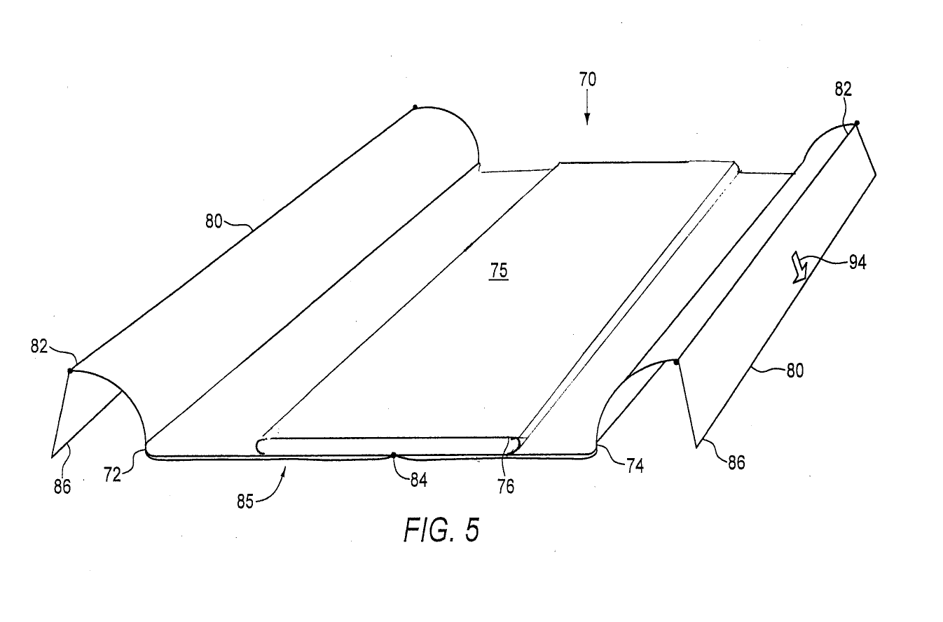

In order to stabilize the shell during the manufacturing and packaging process after the shell is cut to length, and to facilitate the positioning of pad 70 (FIG. 5) within the shell 50 when the mother is replacing it, preferably the shell 50 is manufactured using a more rigid, less pliant material in crotch area 56 by providing a stabilizing material in that area to give greater rigidity to the diaper. For example, the waterproof non-absorbent layer 55 may be replaced with a layer of less pliant material 71 glued to sheet 52 as shown in FIG. 4B. Preferably layer 71 is a layer of flexible, resilient waterproof plastic material such as a closed cell, expanded low-density polyethylene referred to as PE foam from 1 to 5 mm in thickness, preferably about 2 mm thick. Preferably the foam has air pockets and a memory which returns it to its original shape after being squeezed. A suitable material for example is the PE foam underlayment sold by Goodfellow as 2.0 floating foam. Such material can be ecologically friendly in that it is blown with butane. Further the plastic which is used may be degradable, bio-degradable and/or compostable, such as plastic which satisfies ASTM D6400 standards of the Biodegradable Products Institute, including BASF's ECOFLEX resins, and Novamont's Mater-Bi polymers. The material for and thickness of the stabilizer layer 71 is selected so that it is soft and resilient in order to be comfortable for the baby, yet retains its shape when released. The material for and thickness of the stabilizer is selected so that it provides the desired cupping of the shell 52 and opening and separation of the leg gathers 61, 63 to facilitate placement and removal of the insert 70 as described below.

Preferably the foam stabilizer layer 71 extends the full length of the crotch area 56, however shorter lengths, rectangular or other shapes will also be effective. The foam stabilizer layer 71 may have holes cut in it in an appropriate size and pattern in order to permit heat to escape which builds up between the diaper shell and the absorbent insert. For example, it may be provided with an array of small apertures or a few larger apertures. For increased stiffness it may be formed as a textured layer having, for example, a stamped pattern providing a pattern of shallow raised edges or thickenings for strength, such as a cross-hatching, checkerboard or pattern of hollow circles, squares, rectangles, bubbles or other patterns for purposes of rigidity.

FIG. 4B is a cross-section taken along line 4-4 of FIG. 3 (not to scale) showing the first method of attaching a stabilizing layer 71 by being glued to the upper surface of back sheet 52 in the crotch area 56. The foam stabilizer 71 may be fixed in the diaper shell in various other ways besides being glued to the upper surface of back sheet 52 in the crotch area 56. FIG. 4C is a cross-section taken along line 4-4 of FIG. 3 (not to scale) showing a second method of attaching a stabilizing layer 71 by gluing to the outside surface of back sheet 52 in the crotch area 56. FIGS. 4D and 4E are cross-sections taken along line 4-4 of FIG. 3 (not to scale) showing third and fourth methods of attaching a stabilizing layer 71 where a separate inner central sheet 55 is attached to the inner leg gathers 61, 63. The foam stabilizer layer 71 can be glued to the upper or lower surfaces of inner central sheet 55 as in FIGS. 4D and 4E directly or with releasable adhesive strips, or glued directly to the bottom surface of pad 75. In all cases it is preferred that the diaper shell can be manufactured on a current state of the art disposable diaper machine.

Other methods of stiffening and stabilizing the crotch area 56 of shell 50 in the same way as the foam stabilizer layer 71 may be used. A web or net of plastic ridges or ribs may be formed on the inner surface of sheet 52 in the crotch area 56. The additional rigidity in crotch area 56 may also be provided by sandwiching a thin layer of air-laid absorbent or pulp in the manufacturing process between sheets 52 and 55 provided that sheet 55 is liquid impermeable so that the stiffening absorbent layer is not exposed to liquid in the crotch area. For example in the existing disposable diaper structure the amount of pulp 16 in the crotch area could be reduced, super absorbent polymers omitted and cover sheet 15 replaced with a waterproof sheet. Gluing of the pulp pad 16 to the cover sheet 15 would then add extra stability. Again, in order for the diaper to be comfortable and wearable by an infant, the stiffening layer must still have the flexibility in the crotch area comparable to that of current disposable diapers. Similarly an existing disposable diaper structure 10 could be used in the present invention by providing a waterproof membrane over the absorbent pad 16, either secured to the diaper by fixed or releasable adhesive or as a separate liner. The diaper would then serve as the re-usable shell 50 and the pad 16 would function as the stabilizing layer 71.

To make diaper shell 50 re-usable, a disposable absorbent insert 70 (FIG. 5) is provided to be removably inserted in crotch area 56. Insert 70 is sized so that edges 72, 74 fit against leg gathers 61, 63 (or cuffs 62, 64 in FIG. 9) and the insert 70 does not extend beyond the waist areas. Insert 70 has an absorbent pad 75 wherein the absorbent pad is significantly narrower than space between the opposed leg cuffs of the shell 10′. Pad 75 may be manufactured from any of the existing absorbent materials such as fluff, super-absorbent polymer, or fibrous super-absorbent polymer but is preferably an ultra-thin air-laid pulp and polyolefin web with thermally-bonded super-absorbent polymers of the type manufactured by Thermacore™. Such air-laid pads provide greater absorbency with less material and less bulk and thickness. The absorbent pad 75 is preferably wrapped in a layer 76 of non-woven hydrophilic material to keep the skin of the infant from the pad. The pad has protective covers 80 which are formed of hydrophobic material and have elastic members 82 bonded thereto. The protective covers 80 may be secured to pad 75 along a single glue or heat welding line at 84. Covers 80 may also be attached to pad 75 by adhesive on the lower surface of the pad or by other means. The covers may be provided with a releasable adhesive along the lower surface of edges 86 to attach to the interior surface 57 of shell 50.

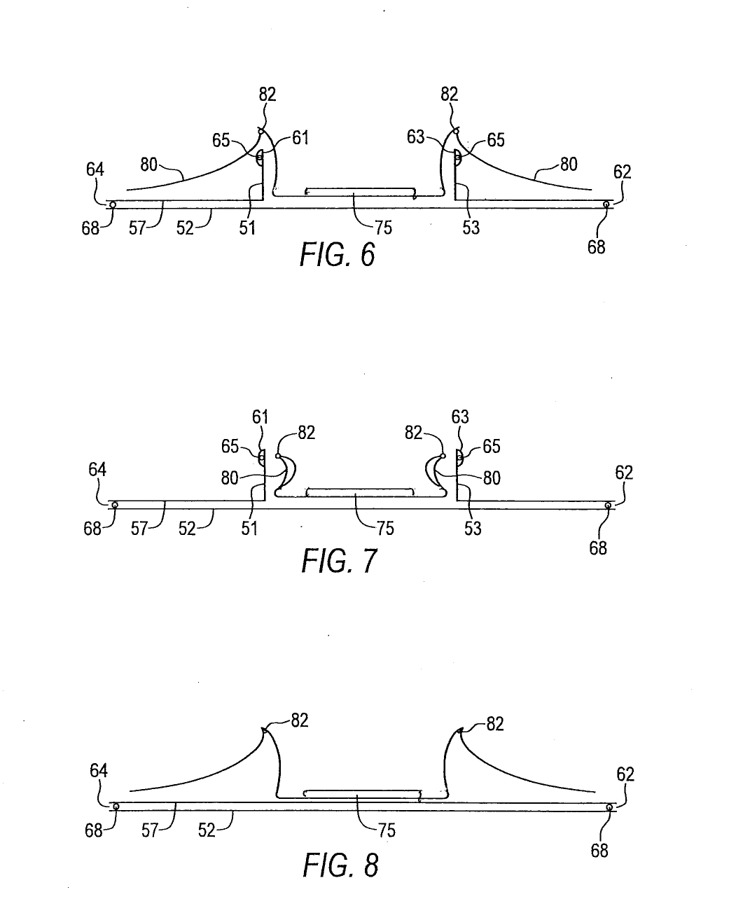

As shown in FIG. 6, by extending the wings over leg gathers 61, 63, the waterproof protective covers 80 protect the shell from being soiled and improve its re-usability. Elastic members 82 serve to elevate the covers 80 above and over the leg gathers 61, 63. Further, when removing and replacing insert 70, covers 80 are used by the mother as handles to remove the insert and then as a wrap to remove and dispose of the contents. Alternatively the mother can place the soiled insert 70 into a bio-degradable plastic disposal bag. Where the shell has no leg gathers, as in the shell 90 in FIG. 9, the protective covers 80 form the leg gathers as in FIG. 8. Where the protective covers 80 are not extended over the leg gathers 61, 63 they can form a second leg gather as shown in FIG. 7 to assist in retaining the waste material in the crotch area 56. Where leg gathers 61, 63 are provided, the pad 75 is significantly narrower than space between the opposed leg gathers 61, 63 along either edge 72, 74 and does not extend beyond the waist areas, as described above, and thereby fits within the channel formed by the leg gathers 61, 63. The invention can also function using a simple narrow pad 75 as the insert between the leg gathers, without wings or elastic. To assist the mother in properly locating the insert, particularly as in the embodiment shown in FIG. 9, the interior of shell 50 may be provided with markings or lines 92 for alignment (FIG. 9) with corresponding markings 94 on insert 70. The lower surface 85 of insert 70 may also be provided with strips of releasable adhesive, protected by removable paper cover strips until in use, in order to assist the mother in better securing the insert 70 in place, for example parallel strips on either side of the lower surface, or the entire lower surface of the pad can be provided with a releasable adhesive protected by a paper cover until removed for use.

Where the covers 80 form a second interior leg gather it can be the same height as leg gathers 61, 63, or will also function if it is higher or lower. Where the re-usable shell 50 has double leg gathers and the covers 80 form a third pair of interior leg gathers, the height of all the leg gathers can be the same. Or alternatively each of the pairs of leg gathers can have different heights or two of the three pairs of leg gathers have the same height, with the third pair having a different height. The tension of the elastic on each pair of leg gathers can similarly be the same as between the re-usable shell and the replaceable insert or one or the other can have a tighter tension.

The insert 70 is manufactured as a separate element and may be packaged and sold separately to the consumer. When sold separately as such, the covers 80 may be folded on top or bottom of pad 75 or completely around the pad, or packaged unfolded. A mother may then choose to insert the insert 70 in a standard disposable diaper to act as a “diaper doubler” either without unfolding covers 80, or by extending the covers 80 to protect the diaper for subsequent re-use to thereby permit re-use of the standard diaper and reduce diaper consumption. Preferably the shell 50 is packaged with an insert 70 already inserted. In that case an insert 70 can be inserted in shell 50 during the manufacturing process as described below, or after the manufacturing of same and prior to packaging. Shell 50 can similarly be packaged for the consumer without an insert 70 inserted therein but rather provided separately. The protective covers 80 can be pre-pressed or folded or packaged in a way that facilitates the covers sitting over interior leg gathers 61, 63 in the most effective position to keep the interior of the shell as clean as possible, such as by folding them under the pad during packaging, or providing double folds over top of the pad as in FIG. 5.

To use the invention where an insert 70 is inserted prior to packaging, the consumer will apply the diaper to the infant in the usual way as with the conventional disposable diaper. Due to the gap between the edges 72, 74 of the insert, the leg gathers 61, 63 of the shell 50 and the absorbent pad 75 which occupies a narrower central region spaced from the leg gathers 61, 63 of the shell, when fitted to the infant a deep central channel is formed which contains waste more effectively. After the pad 75 has been wet or soiled, the insert 70 is removed from shell 50 by grasping covers 80, covers 80 are wrapped around the waste-containing pad 75, secured by the releasable adhesive or placed in a degradable disposal bag, and placed in a waste disposal container or compost. Shell 50 can then be rinsed, and a replacement insert 70 is inserted. After one or more replacement inserts 70 have been similarly used, the shell 50 can be discarded and a new shell 50 and insert 70 used. In this way, the re-usable diaper shell 50 may be re-used once or several times until it also becomes soiled or the adhesive tabs 58 are inoperative. The re-usable diaper shell 50 can be manufactured from more durable materials so that it is can be machine washed and can be manufactured from a durable cloth material, either sewn, glued, sonic welded or other means of securing or fastening using existing disposable diaper manufacturing processes. In this way the diaper shell can be reused at least once and perhaps several times, thereby reducing the consumption of materials and the resulting expense and waste. At the same time, no significant amount of additional work or energy is required by the mother in terms of washing.

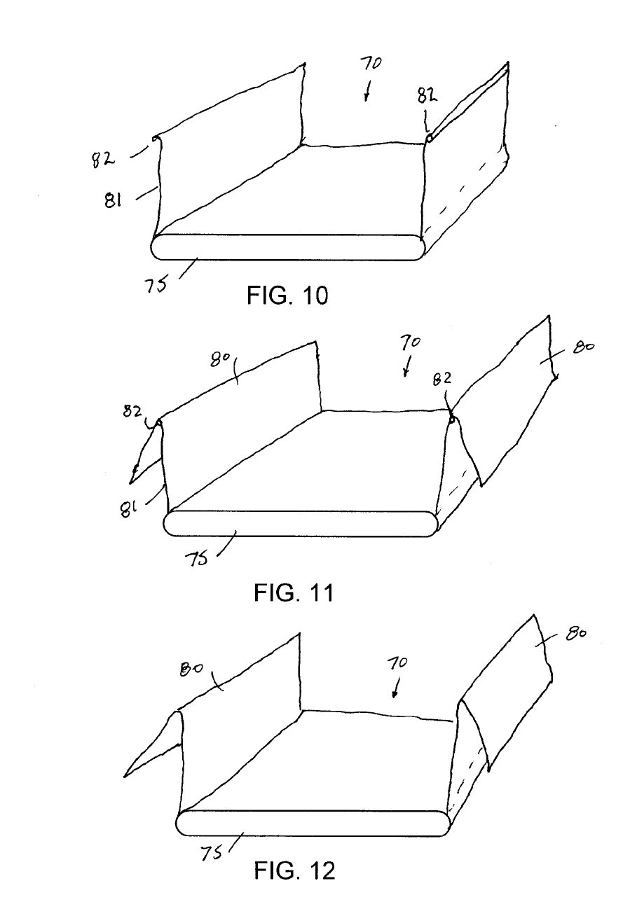

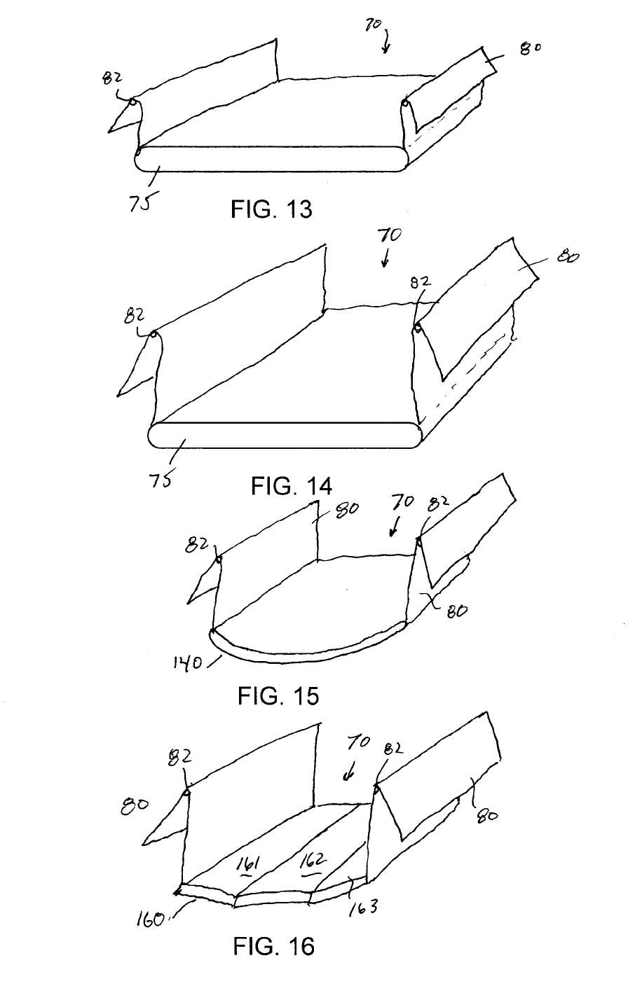

While the foregoing embodiment of disposable insert 70 is preferred, the two-piece diaper system of the invention will also function with different embodiments of the removable insert 70. For example, insert 70 may be produced without wings 80, but with elastic members 82 to form upstanding leg gathers 81 (FIG. 10) or without the elastic members 82 shown in FIG. 10; with wings 80 and elastic members 82 with deeper gathers 81 (FIG. 11); with wings 80 without elastic members 82 (FIG. 12); with short wings 80 to form a shallow central channel (FIG. 13); with tall wings 80 to form a deep central channel (FIG. 14); with a pad 140 that is curved to snugly fit the shell (FIG. 15) and with a pad 160 that has three flat sections 161, 162, 163 joined at angles to snugly fit the shell (FIG. 16). In each case the width of the pad 75, 140, 160 is significantly narrower than the space between the leg gathers of the shell to provide a deep central channel.

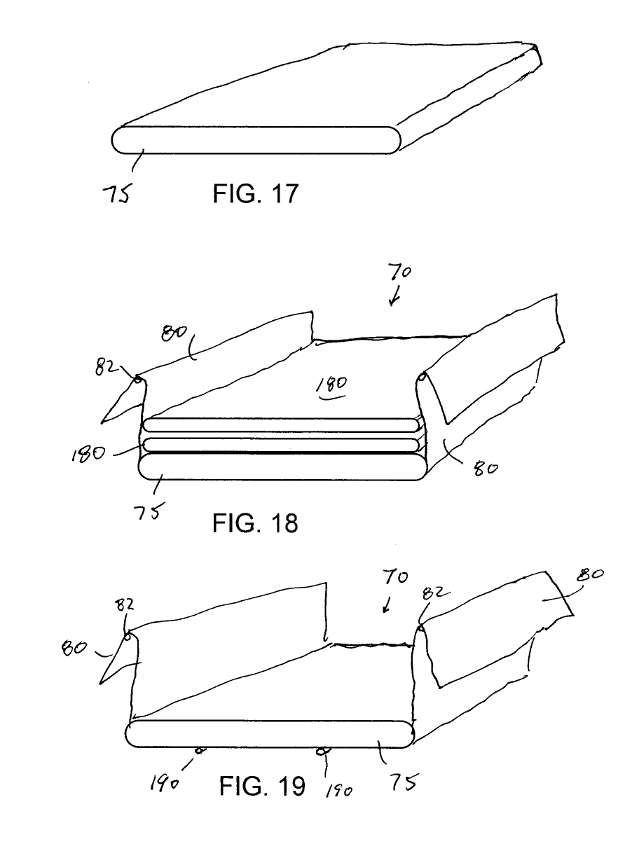

Further embodiments of the removable insert 70 are shown in FIGS. 17, 18 and 19. In each case the width of the pad 75 is effectively significantly narrower than the space between the leg gathers of the shell to provide a deep central channel when worn by the infant. In FIG. 17 the pad 75 has no elastic interior leg gathers or wings. In FIG. 18 the insert is provided with separate removable absorbent pads 180 which can be stacked inside the insert 170 and removed when soiled or wetted. Such multiple pads could be used in any of the foregoing embodiments due to the feature of the deep channel formed in the shell. The separate pads 180 may be provided with holes or channels (not shown) to let fluid penetrate to the lower pads 180, 75 more quickly to increase absorbency. In FIG. 19 elastic members 190 are glued to the underside of pad 75 or insert 70 to assist in providing a lengthwise cupped shape for the insert so that it fits easily into the shell.

In a further embodiment, with reference to FIGS. 5 and 6, since back sheet 85 of the insert 70 and wings 80 may be formed of hydrophobic material, the back sheet 52 of the re-usable shell 50 may be formed of a breathable material, preferably a non-woven polyester or polypropylene or Novamont's Mater-Bi polymers. The outer edges of the shell 50 may still be formed of a waterproof plastic film, but by having a breathable material in the central back sheet area of the shell, the build-up of heat in that area is reduced and frequency diaper rash diminished. Using a second or multiple stackable separate pads 180 as described above in combination with this embodiment increases the absorbency within the insert 70 to reduce the likelihood of any leakage through the breathable back sheet.

Existing machines for manufacturing disposable diapers, such as those manufactured by Fammeccanica, PCMC, Joa and Cellulose Converting Equipments, utilize a continuous line in which a ribbon of the absorbent pulp is formed and shaped, and fed on a supporting surface such as tissue, the outer poly sheet is unwound from below the pulp line and adhesive and leg elastic are applied to it. The absorbent layer is then applied to the poly sheet, and a non-woven topsheet is applied over the pulp layer and bonded to the poly back sheet, the elasticized leg gathers and tape tabs are applied and the diapers are then cut and folded. In the preferred method of manufacturing the present invention, the shell 50 is manufactured in the same manner as conventional diapers but without including any sandwiched absorbent layer, while absorbent insert 70 is manufactured separately. The inserts 70 are preferably manufactured from air laid, thermal bonded air laid Super Absorbent Polymers technology roll stock. The non-woven pad cover and waterproof protective covers 80 with elastic members 82 are attached to the pad, and the inserts are cut to length, folded and packaged. The initial inserts 70 can be inserted into shells 50 during the manufacturing process by providing the finished but uncut inserts 70 on a continuous roll which is fed into the line of shells on the diaper line prior to cutting of the individual diapers. Similarly the pre-cut stacked pads 180 can fed into the line of shells on the diaper line prior to cutting of the individual diapers.

It is possible with the present invention to obtain the same results from one shell and three or more inserts as is obtained from three or more current disposable diapers. The shell 50, being non-absorbent and waterproof can be rinsed with a soap cleaning solution and a few drops of disinfectant if desired, or washed by hand or machine for re-use after each diaper change. The present invention therefore assists in the goal of reducing a consumer's carbon footprint by reducing, re-using and recycling the plastic and hydrocarbon-based material. This is accomplished by a) reducing the consumption of materials and energy through thinner, more efficient absorption pads and multiple uses of the diaper shell, thereby reducing shipping costs, landfill and energy required for manufacturing, laundering and/or disposal and hauling away; b) the diaper shell is re-used, possibly multiple times, thereby providing an efficient way to reduce the use of plastic; and c) the economies of the system permit more expensive, biodegradable materials to be used and still permit the product to compete with non-biodegradable materials. Further the diaper system can be manufactured using existing conventional disposable diaper machines, eliminating the need for expensive new equipment. The carbon credits earned by the consumer on purchasing the present diaper system can be translated into a discount to the consumer retail price in exchange for the manufacturer retaining the credits to permit the manufacturer to deal with or trade in a large volume of carbon credits.

As will be apparent to those skilled in the art in the light of the foregoing disclosure, many alterations and modifications are possible in the practice of this invention without departing from the spirit or scope thereof. While the invention has been described in the context of an infant diaper it is also applicable to adult incontinence diapers. Thus while a number of exemplary aspects and embodiments have been discussed above, those of skill in the art will recognize certain modifications, permutations, additions and sub-combinations thereof.

What is claimed is:

1. A diapering system comprising:
a) a disposable re-usable diaper shell comprising:
i) a pliant non-absorbent, liquid impervious body for removable fitting to the wearer, forming an interior and an exterior surface, a front and back portion, opposed side edges and a crotch area when so fitted;
ii) opposed, non-absorbent liner portions secured along the opposed side edges of the body and forming opposed elastically contractible leg cuffs extending between the front and back portion, the opposed elastically contractible leg cuffs thereby defining a central channel region between the opposed leg cuffs extending between the front and back portion, the central channel region being without absorbent material which is exposed to contact with liquid in the crotch area and comprising in the central channel region flexible means for stabilizing said diaper shell; and
b) a removable, replaceable absorbent insert comprising an absorbent pad comprising a body of absorbent material having a non-woven hydrophilic covering, said insert being sized and shaped to be retained adjacent the interior surface of the diaper shell in the central channel region and wherein the absorbent pad is significantly narrower than the space between the opposed leg cuffs whereby when fitted to the wearer said central channel region is significantly deeper than the height of said opposed leg cuffs.

2. The diapering system of claim 1 wherein said removable, replaceable absorbent insert comprises attached hydrophobic covers extending from opposed sides of said absorbent pad and extending over the interior surface of said pliant non-absorbent, liquid impervious body when in operative position.

3. The diapering system of claim 2 wherein the hydrophobic covers have elongated elastic members secured thereto to form leg gathers when in operative position.

4. The diapering system of claim 1 wherein means for stabilizing comprises a layer of non-absorbent plastic material secured to the non-absorbent, liquid impervious interior surface in said crotch area of said body.

5. The diapering system of claim 1 wherein said pliant, non-absorbent, liquid impervious body comprises a plastic film.

6. The diapering system of claim 4 wherein said layer of non-absorbent plastic material comprises a polyethylene foam.

7. The diapering system of claim 1 wherein said means for stabilizing comprises a layer of absorbent material covered by a waterproof layer whereby said absorbent material is not exposed to contact with liquid in the crotch area.

8. The diapering system of claim 1 wherein said means for stabilizing comprises a layer of non-absorbent plastic material secured to the non-absorbent, liquid impervious outer surface in said crotch area of said body.

9. The diapering system of claim 5 wherein said plastic film is compostable.

10. The diapering system of claim 5 wherein said plastic film comprises a biodegradable film.

11. A disposable re-usable diaper shell comprising a pliant non-absorbent, liquid impervious body for removable fitting to the wearer, forming an interior and an exterior surface, a front and back portion, opposed side edges and a crotch area when so fitted, said diaper shell comprising opposed, non-absorbent liner portions secured along said opposed side edges of said body and forming opposed elastically contractible leg cuffs extending between said front and back portion, said opposed elastically contractible leg cuffs thereby defining a central channel region between said opposed leg cuffs extending between said front and back portion, the central channel region being without absorbent material which is exposed to contact with liquid in the crotch area and comprising in the central channel region flexible means for stabilizing said diaper shell, said central region being adapted for removably receiving an absorbent pad in said central channel region and wherein the absorbent pad is significantly narrower than the space between the opposed leg cuffs whereby when fitted to the wearer said central channel region is significantly deeper than the height of said opposed leg cuffs.

12. The disposable re-usable diaper shell of claim 11 wherein said means for stabilizing comprises a layer of non-absorbent plastic material secured to the non-absorbent, liquid impervious interior surface in said crotch area of said body.

13. The disposable re-usable diaper shell of claim 11 wherein said pliant, non-absorbent, liquid impervious body comprises a plastic film.

14. The diaper shell of claim 12 wherein said layer of non-absorbent plastic material comprises a polyethylene foam.

15. The disposable re-usable diaper shell of claim 11 wherein said means for stabilizing comprises a layer of absorbent material covered by a waterproof layer whereby said absorbent material is not exposed to contact with liquid in the crotch area.

16. The disposable re-usable diaper shell of claim 11 wherein said means for stabilizing comprises a layer of non-absorbent plastic material secured to the non-absorbent, liquid impervious outer surface in said crotch area of said body.

17. The diaper shell of claim 13 wherein said plastic film is compostable.

18. The diaper shell of claim 13 wherein said plastic film comprises a biodegradable film.

19. A removable, replaceable absorbent diaper insert comprising an absorbent pad comprising a body of absorbent material having a non-woven hydrophilic covering, said insert being sized and shaped to be retained adjacent the interior surface of a diaper shell in the central region thereof, wherein said removable, replaceable absorbent insert comprises attached hydrophobic covers extending from opposed sides of said absorbent insert and adapted to extend over the interior surface of a diaper shell having opposed leg cuffs and wherein the absorbent pad is significantly narrower than the space between the opposed sides of said absorbent insert whereby when said diaper shell is fitted to the wearer it forms a central channel region is significantly deeper than the height of said opposed leg cuffs.

20. A removable, replaceable absorbent diaper insert of claim 19 wherein the hydrophobic covers have elongated elastic members secured thereto to form leg gathers when in operative position.

21. In combination in a package, the diaper shell of claim 11 and a plurality of removable, replaceable absorbent diaper inserts adapted to be removably placed in said central channel region of said diaper shell.

22. A method of manufacturing a diaper shell without absorbent material which is exposed to contact with liquid in the crotch area thereof and adapted for removably inserting an absorbent pad, said method comprising bonding in a continuous process a sheet of non-woven hydrophobic liner material to a sheet of liquid impervious backsheet material, and cutting said bonded sheets to form a disposable diaper shell comprising a non-absorbent, liquid impervious body and a non-absorbent liner having opposed elastically contractible leg cuffs, said diaper shell being without absorbent material in said crotch area which is exposed to contact with liquid in the crotch area and comprising in the central channel region flexible means for stabilizing said diaper shell and being adapted for removably inserting an absorbent pad in said central channel region, said stabilizing means thereby stabilizing said disposable shell for insertion of said absorbent pad, and wherein the absorbent pad is significantly narrower than the space between the opposed leg cuffs whereby when fitted to the wearer said central channel region is significantly deeper than the height of said opposed leg cuffs.

23. The method of claim 22 wherein said means for stabilizing comprises a layer of non-absorbent plastic material secured to the non-absorbent, liquid impervious interior surface in said crotch area of said body.

24. The method of claim 22 wherein said means for stabilizing comprises a layer of absorbent material covered by a waterproof layer whereby said absorbent material is not exposed to contact with liquid in the crotch area.

25. The method of claim 22 wherein said means for stabilizing comprises a layer of non-absorbent plastic material secured to the non-absorbent, liquid impervious outer surface in said crotch area of said body.
